(19) United States
(12) Patent Application Publication
(10) Pub. No.: US 2022/0354378 A1
(43) Pub. Date: Nov. 10, 2022

(54) NEURAL INTERVENTIONAL MAGNETIC RESONANCE IMAGING APPARATUS

(71) Applicant: Neuro42 Inc., Oakland, CA (US)

(72) Inventors: Aleksandar NACEV, San Francisco, CA (US); Amit VOHRA, Rocklin, CA (US); Dinesh KUMAR, Roseville, CA (US); Nikhil SADWANI, Alameda, CA (US); Nio ANDERSON, San Francisco, CA (US)

(73) Assignee: Neuro42 Inc., Oakland, CA (US)

(21) Appl. No.: 17/662,189

(22) Filed: May 5, 2022

Related U.S. Application Data
(60) Provisional application No. 63/184,748, filed on May 5, 2021.

(51) Int. Cl. A61B 5/055; G01R 33/34; G01R 33/3415; G01R 33/38

(52) U.S. Cl. CPC A61B 5/055; G01R 33/34; G01R 33/3415; G01R 33/3806; A61B 2090/374

(57) ABSTRACT

A magnetic resonance imaging (MRI) apparatus is disclosed. The MRI apparatus includes a plurality of magnetic elements affixed in a Halbach dome structure. The Halbach dome structure defines an access aperture configured to allow access to the patient's head to enable neural intervention and defines a patient opening configured to receive a patient's head. In various aspects, the Halbach dome comprises a plurality of access apertures and/or gaps that may be adjustable in size.

### CROSS-REFERENCE TO RELATED APPLICATIONS

This application claims the benefit of priority under 35 U.S.C. § 119(e) to U.S. Provisional Patent Application No. 63/184,748, titled NEURAL INTERVENTIONAL MAGNETIC RESONANCE IMAGING APPARATUS, filed May 5, 2021, the entire disclosure of which is incorporated by reference herein.

### BACKGROUND

The present disclosure relates to magnetic resonance imaging (MRI), medical imaging, medical intervention, and surgical intervention. MRI devices are often large and complex machines that create significant constraints on the feasibility of performing surgical interventions. These restrictions come in many forms including limited physical access to the patient either by a surgeon or robot and limitations in the usage of electrical and mechanical components within proximity to the scanner. Since modern MRI scanners are not designed to enable surgical guidance, these limitations are inherent in the underlying design of the system and are difficult to overcome.

### SUMMARY

In one aspect, the present disclosure describes a magnetic resonance imaging apparatus comprising: a structural housing configured as a dome shape, wherein the structural housing is configured to receive a patient's head at the base of the dome, and wherein the structural housing defines an access aperture configured to allow access to the patient's head to enable neural intervention; and a plurality of magnetic elements configured in a Halbach array, wherein the plurality of magnetic elements are permanently affixed to an interior surface of the structural housing.

In another aspect, the present disclosure describes a magnetic resonance imaging apparatus comprising: a structural housing configured as a dome shape, wherein the structural housing is configured to receive a patient's head at the base of the dome; the structural housing comprises a plurality of wedges defining an access aperture configured to allow access to the patient's head to enable neural intervention; and a plurality of magnetic elements configured in a Halbach array, wherein the plurality of magnetic elements are permanently affixed to an interior surface of the structural housing.

In yet another aspect, the present disclosure describes a neural intervention system, comprising: a magnetic resonance imaging system comprising: a magnetic resonance imaging apparatus further comprising a plurality of magnetic elements in Halbach dome, wherein the Halbach dome defines an access aperture configured to allow access to the patient's head to enable neural intervention; and a guided robotic system comprising: a robotic arm wherein the guided robotic system is configured to guide the robotic arm through the access aperture of the Halbach dome for neural intervention.

### BRIEF DESCRIPTION OF THE DRAWINGS

The various aspects described herein, both as to organization and methods of operation, together with further objects and advantages thereof, may best be understood by reference to the following description, taken in conjunction with the accompanying drawings as follows.

FIG. 1 illustrates a Halbach cylinder having a wall structure defining an access aperture configured to receive a patient's head therein, in accordance with at least one aspect of the present disclosure.

FIG. 2 illustrates a C-shaped yoked dipole defining an access aperture, in accordance with at least one aspect of the present disclosure.

FIG. 3 illustrates a full yoked dipole defining and access aperture, in accordance with at least one aspect of the present disclosure.

FIG. 4 is a control schematic for a magnetic resonance imaging system, in accordance with at least one aspect of the present disclosure.

FIG. 5 illustrates a Halbach dome defining an access aperture in the form of a hole, where the dome is configured to receive a patient's head and the access aperture is configured to allow access to the patient's head to enable neural intervention, in accordance with at least one aspect of the present disclosure.

FIG. 6 is a cross-sectional view of the Halbach dome with the access hole shown in FIG. 5 illustrated, in accordance with at least one aspect of the present disclosure.

FIG. 7 illustrates a Halbach dome defining an access aperture in the form of a gap, where the Halbach dome is configured to receive a patient's head and the access gap is configured to allow access to the patient's head to enable neural intervention, in accordance with at least one aspect of the present disclosure.

FIG. 8 is a cross-sectional view of the Halbach dome with the access gap shown in FIG. 7 illustrated, in accordance with at least one aspect of the present disclosure.

FIG. 9 shows illustrates a MRI system used in connection with a robotic system, in accordance with various embodiments.

FIGS. 10A, 10B, and 10C illustrate line simulations of an Halbach dome without an access aperture, including an isometric view, a bottom view, and a top view, respectively, in accordance with at least one aspect of the present disclosure.

FIGS. 11A, 11B, and 11C illustrate magnetic flux density B relative to the x, y, and z directions, respectively, of the Halbach dome shown in FIGS. 10A, 10B, and 10C, in accordance with at least one aspect of the present disclosure.

FIGS. 12A, 12B, and 12C illustrate line simulations of an Halbach dome defining an access aperture having an access aperture diameter of D≈10 cm, including an isometric view, a bottom view, and a top view, respectively, in accordance with at least one aspect of the present disclosure.

FIGS. 13A, 13B, and 13C illustrate magnetic flux density B relative to the x, y, and z directions, respectively, of the Halbach dome shown in FIGS. 12A, 12B, and 12C, in accordance with at least one aspect of the present disclosure.

FIGS. 14A, 14B, and 14C illustrate line simulations of an Halbach dome defining an access aperture having a access aperture diameter of D≈16 cm, including an isometric view, a bottom view, and a top view, respectively, in accordance with at least one aspect of the present disclosure.

FIGS. 15A, 15B, and 15C illustrate magnetic flux density B relative to the x, y, and z directions, respectively, of the Halbach dome shown in FIGS. 14A, 14B, and 14C, in accordance with at least one aspect of the present disclosure.

FIGS. 16 and 17 are views of a Halbach dome with an access gap having an access gap width of W_{gap}≈10 cm, in accordance with at least one aspect of the present disclosure.

FIGS. 18A, 18B, and 18C illustrates magnetic flux density B relative to the x, y, z directions, respectively, of the Halbach dome shown in FIGS. 16 and 17, in accordance with at least one aspect of the present disclosure.

FIG. 19 is a schematic of an Halbach dome comprising a plurality of wedges, in accordance with at least one aspect of the present disclosure.

FIG. 20 is a top view of the Halbach dome of FIG. 19, in accordance with at least one aspect of the present disclosure.

FIG. 21 is an isometric view of an Halbach dome defining a plurality of access apertures, in accordance with at least one aspect of the present disclosure.

FIG. 22 is an isometric view of an Halbach dome defining a plurality of access apertures and an adjustable gap, in accordance with at least one aspect of the present disclosure.

FIG. 23 illustrates a scanning system including a Halbach dome defining an access aperture, in accordance with at least one aspect of the present disclosure.

Corresponding reference characters indicate corresponding parts throughout the several views. The exemplifications set out herein illustrate various disclosed embodiments, is one form, and such exemplifications are not to be construed as limiting the scope thereof in any manner.

### DETAILED DESCRIPTION

Before explaining various aspects of neural interventional magnetic resonance imaging device in detail, it should be noted that the illustrative examples are not limited in application or use to the details of construction and arrangement of parts illustrated in the accompanying drawings and description. The illustrative examples may be implemented or incorporated in other aspects, variations and modifications, and may be practiced or carried out in various ways. Further, unless otherwise indicated, the terms and expressions employed herein have been chosen for the purpose of describing the illustrative examples for the convenience of the reader and are not for the purpose of limitation thereof. Also, it will be appreciated that one or more of the following-described aspects, expressions of aspects, and/or examples, can be combined with any one or more of the other following-described aspects, expressions of aspects and/or examples.

Various aspects are directed to neural interventional magnetic resonance imaging (MRI) devices that allows for the integration of surgical intervention and guidance with an MRI. This includes granting physical access to the area around the patient as well as access to the patient's head with access apertures. In addition, the neural interventional magnetic resonance imaging device should allow for the usage of robotic guidance tools and traditional surgical implements.

There are many possible configurations of neural interventional magnetic resonance imaging devices that can achieve improved access for surgical intervention. These configurations build upon the two main designs as taught by Cooley et al. (e.g. Cooley, C. Z., Haskell, M. W., Cauley, S. F., Sappo, C., Lapierre, C. D., Ha, C. G., Stockmann, J. P., & Wald, L. L. (2018). Design of sparse Halbach magnet arrays for portable MRI using a genetic algorithm. IEEE transactions on magnetics, 54(1), 5100112, commonly known as the Halbach cylinder and the Halbach dome, each of which is incorporated herein by reference. Possible configurations are shown in the following figures. The article “Design of sparse Halbach magnet arrays for portable MRI using a genetic algorithm” by Cooley et al., published in IEEE transactions on magnetics, 54(1), 5100112 in 2018, is incorporated by reference herein in its entirety.

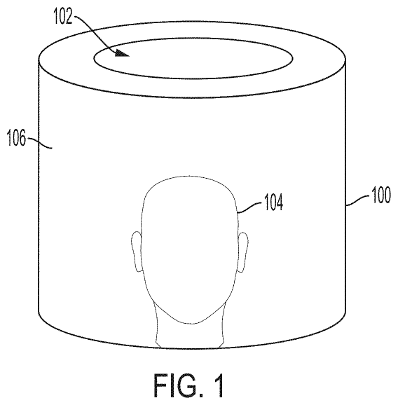

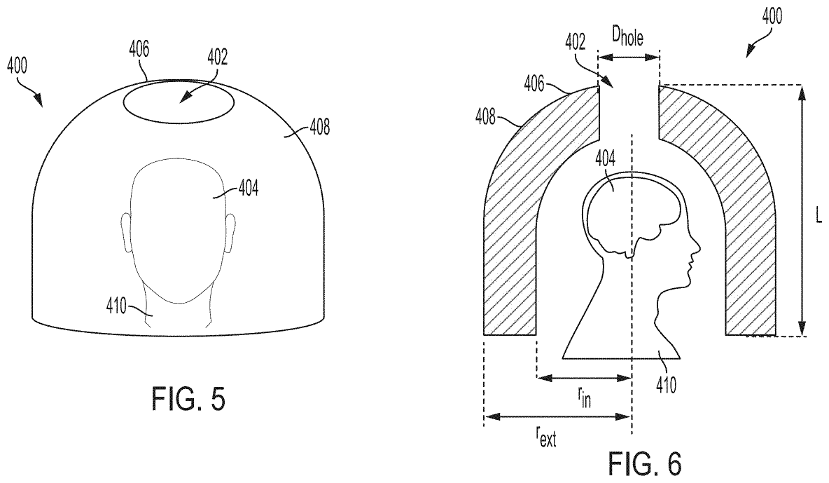

FIG. 1 illustrates a Halbach cylinder 100 having a wall structure 106 defining an access aperture 102 configured to receive a patient's head 104 therein, in accordance with at least one aspect of the present disclosure. Additional access apertures as described herein with reference to FIGS. 5-8, for example, may be defined in the wall structure 106 of the cylinder 100. The Halbach cylinder comprises a plurality of magnetic elements in a magnetic assembly that is configured in a Halbach array. The Halbach array may be configured, based on the configuration of the plurality of magnetic elements, such that a main magnetic flux density, Bₒ, is generated by the plurality of magnetic elements. In various aspect, the magnetic elements comprise permanent magnets or electro-permanent magnets.

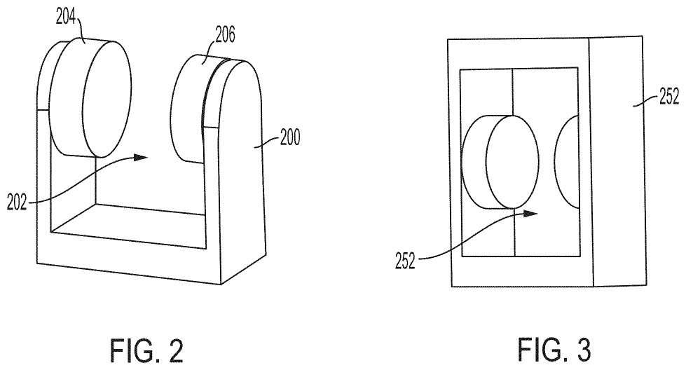

FIG. 2 illustrates a C-shaped yoked dipole 200 defining an access aperture 202, in accordance with at least one aspect of the present disclosure. The C-shaped yoked dipole 200 comprises a first magnetic element 204 and a second magnetic element 206, where a main magnetic field Bₒ extends across the gap in the direction of the second magnetic element 206. In various aspects, each of the first magnetic element 204 and the second magnetic element 206 may be configured as a single magnetic polarity or may be configured as a plurality of magnetic elements in a magnetic assembly. The C-shaped yoked dipole 200 provides greater access to a patient between the first magnetic element 204 and the second magnetic element 206, compared to the Halbach cylinder 100. However, the C-shaped yoked dipole 200 is limited in the main magnetic field Bₒ strength, size, and homogeneity compared to the Halbach cylinder 100.

FIG. 3 illustrates a full yoked dipole 250 defining an access aperture 252, in accordance with at least one aspect of the present disclosure. The full yoked dipole 250 resolves some of the limitations by of the C-shaped yoked dipole 200 but reduces the access aperture in the process.

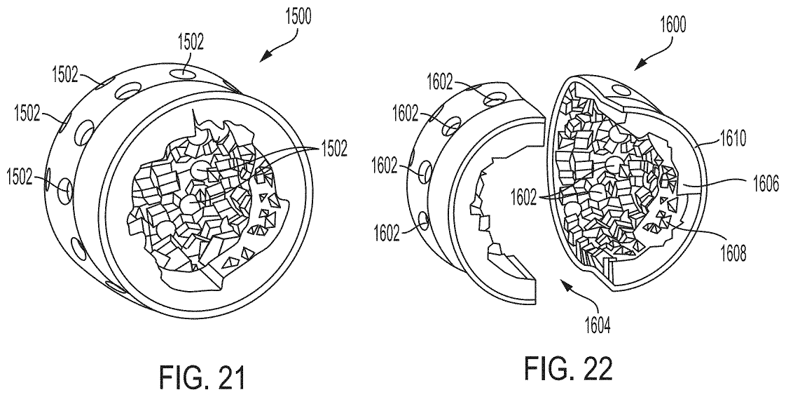

The present disclosure further describes a Halbach dome that provides a configurable dome shape based on several factors including main magnetic field Bₒ strength, field size, field homogeneity, device size, device weight, and access to the patient for neural intervention. In various aspects, the Halbach dome comprises an exterior radius rₑₓₜ and interior radius rᵢₙ at the base of the dome. The Halbach dome may comprise an elongated cylindrical portion that extends from the base of the dome. In one aspect, the elongated cylindrical portion comprises the same exterior radius and interior radius as the base of the dome and continues from the base of the dome at a predetermined length, at a constant radius (see FIGS. 5-7). In another aspect, the elongated cylindrical portion comprises a different exterior radius and interior radius than the base of the dome (see FIGS. 17 and 21). The different exterior radius and interior radius of the elongated cylindrical portion merges with the base radii in a transitional region.

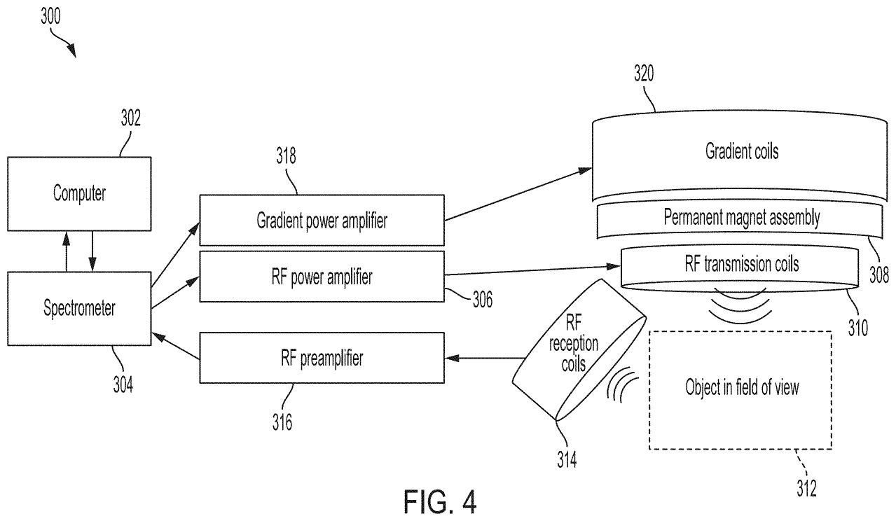

FIG. 4 shows a control schematic for an MRI system 300. For example, the imaging system 300 includes a magnet assembly 308, which can be similar to the Halbach cylinder 100 or Halbach domes (see, e.g., FIGS. 5-8, 10, 12, 14, and 19-23) in various instances. The imaging system 300 also includes RF transmission coils 310 and RF reception coils 314. In various aspects, the RF transmission coils 310 and/or the RF reception coils can also be positioned in the housing of an MRI scanner and, in certain instances, the RF transmission coils 310 and the RF reception coils 314 can be combined into integrated Tx/Rx coils. The system 300 also includes gradient coils 320, which are configured to generate gradient fields to facilitate imaging of the object in the field of view 312.

The MRI system 300 also includes a computer 302, which is in signal communication with a spectrometer 304, and is configured to send and receive signals between the computer 302 and the spectrometer 304. In various aspect, the main magnetic field Bₒ, generated by the magnetic assembly 308, extends away from the magnetic assembly 308 and away from the RF transmission coils 310 into the field of view 312. The field of view 312 contains an object that is being imaged by the MRI system 300.

During the imaging process, the main magnetic field Bₒ extends into the field of view 312. The direction of the effective magnetic field (B₁) changes in response to the RF pulses and associated electromagnetic fields from the RF transmission coils 310. For example, the RF transmission coils 310 may be configured to selectively transmit RF signals or pulses to an object in the field of view, e.g. tissue. These RF pulses may alter the effective magnetic field experienced by the spins in the sample (e.g. patient tissue).

Moreover, when the object in the field of view 312 is excited with RF pulses from the RF transmission coils 310, the precession of the object results in an induced electric current, or MR current, which is detected by the RF reception coils 314. The RF reception coils 314 can send the excitation data to an RF preamplifier 316. The RF preamplifier 316 can boost or amplify the excitation data signals and send them to the spectrometer 304. The spectrometer 304 can send the excitation data to the computer 302 for storage, analysis, and image construction. The computer 302 can combine multiple stored excitation data signals to create an image, for example.

From the spectrometer 304, signals can also be relayed to the RF transmission coils 310 via an RF power amplifier 306, and to the gradient coils 320 via a gradient power amplifier 318. The RF power amplifier 306 amplifies the signal and sends it to RF transmission coils 310. The gradient power amplifier 318 amplifies the gradient coil signal and sends it to the gradient coils 320.

FIG. 5 illustrates a Halbach dome 400 defining an access aperture in the form of a hole 402, where the dome 400 is configured to receive a patient's head 404 and the access hole 402 is configured to allow access to the patient's head 404 to enable neural intervention, in accordance with at least one aspect of the present disclosure. The Halbach dome 400 configuration can be built with a single access hole 402 at the top side 406 of the dome 400 or multiple access apertures 402 around the structure 408 of the dome 400. This configuration allows for access to the top of the skull while minimizing the impact to the magnetic field. The diameter D of the access hole 402 may be small (about an 2.54 cm) or very large (substantially the exterior rₑₓₜ diameter of the dome 400). However, as the access hole 402 becomes larger, the dome 400 begins to resemble a Halbach cylinder 100 design, as shown in FIG. 1. The hole 402 is not limited to being at the apex of the dome 400, and it can be placed anywhere on the surface or structure 408 of the dome 400. The entire dome 400 system can be rotated so that the access hole 402 can be co-located with a desired physical location on the patient 410.

FIG. 6 is a cross-sectional view of the Halbach dome 400 with the access hole 402 shown in FIG. 5, in accordance with at least one aspect of the present disclosure. FIG. 6 shows relative dimensions of the Halbach dome 400 defining an access hole 402 such as diameter D of the access hole 402, length L of the dome 400 and exterior radius rₑₓₜ and interior radius rᵢₙ of the dome 400. The Halbach dome 400 comprises a plurality of magnetic elements that are configured in a Halbach array and make up a magnetic assembly. The plurality of magnetic elements may be enclosed by the exterior radius rₑₓₜ and interior radius rᵢₙ in a housing. In one aspect, example dimensions may be defined as:

rᵢₙ=19.3 cm;

rₑₓₜ=23.6 cm;

L=38.7 cm; and

2.54 cm≤D<19.3 cm.

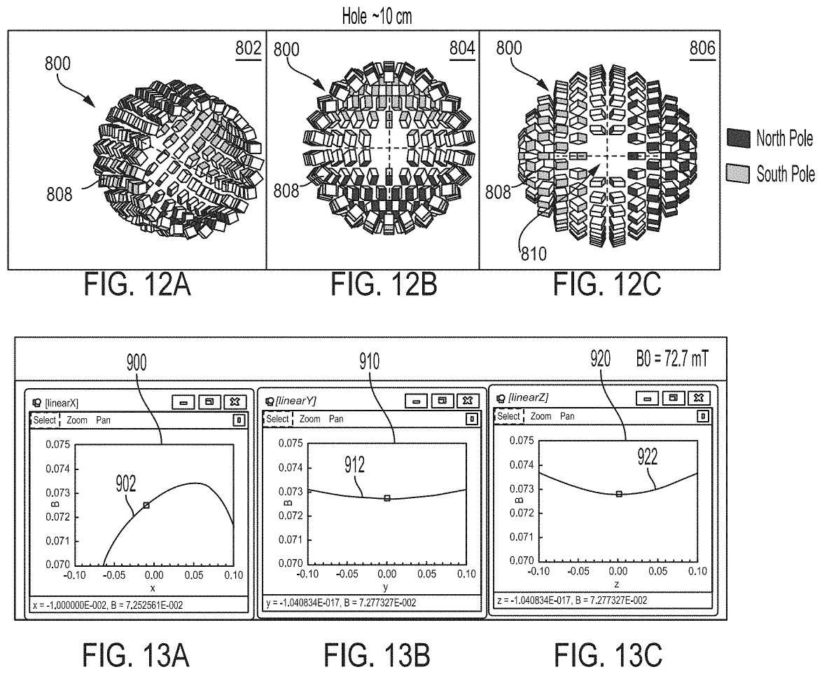

Based on the above example dimensions, a Halbach dome 400 with an access aperture 402 may be configured with a magnetic flux density Bₒ of around 72 mT, and an overall mass of around 35 kg, see FIGS. 12 and 13. It will be appreciated that the dimensions may be selected based on particular applications to achieve a desired magnetic flux density Bₒ and geometry of the neural intervention access hole 402.

In various aspects, the Halbach dome 400 may be configured to define multiple access apertures 402 placed anywhere around the structure 408 of the dome 400. These multiple access apertures 402 may be configured to allow for access to the patient's head 404 using tools (e.g., surgical tools) or a robot.

In various aspects, the access hole 402 may be configured to be adjustable. The adjustable configuration may provide the ability for the access hole 402 to be adjusted using either a motor, mechanical assist, or a hand powered system with an iris configuration, for example, to adjust the diameter D of the access hole 402. This would allow for the attachment of the imaging device dome 400 in a configuration with no access hole 402, conducting an imaging scan, and then adjusting the configuration of the imaging device dome to include the access hole 402 to enable a surgical intervention. The access hole 402 can range from a width Dₕₒₗₑ of 2.54 cm in size to the interior diameter rᵢₙₜ of the dome 400. In an aspect where the Dₕₒₗₑ is equal to the interior diameter rᵢₙₜ, the Halbach dome 400 would be configured similar to Halbach cylinder 100.

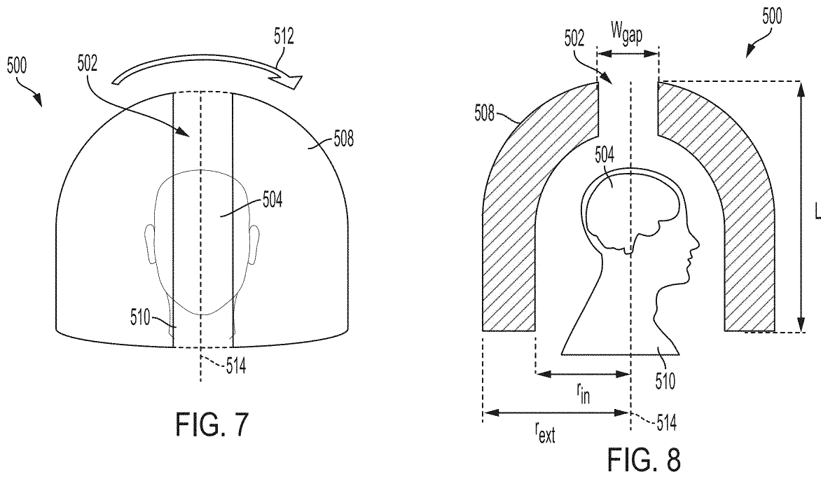

FIG. 7 illustrates a Halbach dome 500 defining an access aperture in the form a gap 502, where the dome 500 is configured to receive a patient's head 504 and the access gap 502 is configured to allow access to the patient's head 504 to enable neural intervention, in accordance with at least one aspect of the present disclosure. The Halbach dome 500 with access gap 502 configuration can define a single large access gap 502, or multiple access gaps 502, defined by the structural housing 508 of the dome 500. This access gap 502 is shown to extend through the longitudinal axis 514 of the patient 510 as if it bisected the patient's head 504 through the nose to the back of the head 504. Similar to the access hole 402 shown in FIGS. 1 and 6, the access gap 502 shown in FIG. 7 can range from a width W_{gap} of 2.54 cm in size up to the interior diameter (2×rᵢₙₜ) of the dome 500. The access gap 502 does not need to be in the plane depicted in FIG. 7, and can be placed in any orientation within the magnet dome 500 of the device. The entire dome 500 system may be rotatable, as shown by the arrow 512, so that the access gap 502 can be co-located with a desired physical location on the patient 510.

FIG. 8 is a cross-sectional view of the Halbach dome 500 with access gap 502 shown in FIG. 7, in accordance with at least one aspect of the present disclosure. FIG. 7 shows relative dimensions of the Halbach dome 500 with access gap 502 such as width W_{gap} of the access gap 502, length L of the dome 500, exterior radius rₑₓₜ, and interior radius rᵢₙ of the dome 500. In one aspect, example dimensions may be defined as:

rᵢₙ=19.3 cm;

rₑₓₜ=23.6 cm;

L=38.7 cm; and

2.54 cm≤W_{gap}<19.3 cm.

Based on the above example dimensions, a Halbach dome 500 with an access gap 502 may be configured with a magnetic flux density Bₒ of around 72 mT, and an overall mass of around 35 kg. It will be appreciated that the dimensions may be selected based on particular applications to achieve a desired magnetic flux density Bₒ and neural intervention access gap 502.

In various aspects, the structural housing 508 of the dome 500 may be configured to define multiple gaps 502 around the structural housing 508 of the dome 500. These multiple access gaps 502 may be configured to allow for access to the patient's head 504 using tools (e.g., surgical tools) or a robot.

In various aspects, the access gap 502 may be adjustable. The adjustable configuration may provide the ability for the access gap 502 to be adjusted using either a motor, mechanical assist, or a hand powered system. This would allow for the attachment of the imaging device dome 500 in a configuration with no access gap 502, conducting an imaging scan, and then adjusting the configuration of the imaging device dome to include the access gap 502 to enable a surgical intervention. Additionally, the adjustment of the access gap 502 may allow the magnetic field to be shimmed according to a particular imaging need or target location on the patient. In various aspect, the length of the access gap may extend from the center of the crown of the Halbach dome, along the surface distance of the exterior, to the base of the dome.

With reference to FIGS. 5-8, in various aspects, a Halbach dome may be configured with a combination of access holes 402 and gaps 502.

Further, with reference back to FIG. 1, in various aspects, the Halbach cylinder 100 may be configured with multiple access apertures defined by the wall structure 106 of the cylinder 100 such as the access holes 402 shown in FIGS. 5 and 6 and/or the access gaps 502 shown in FIGS. 7 and 8.

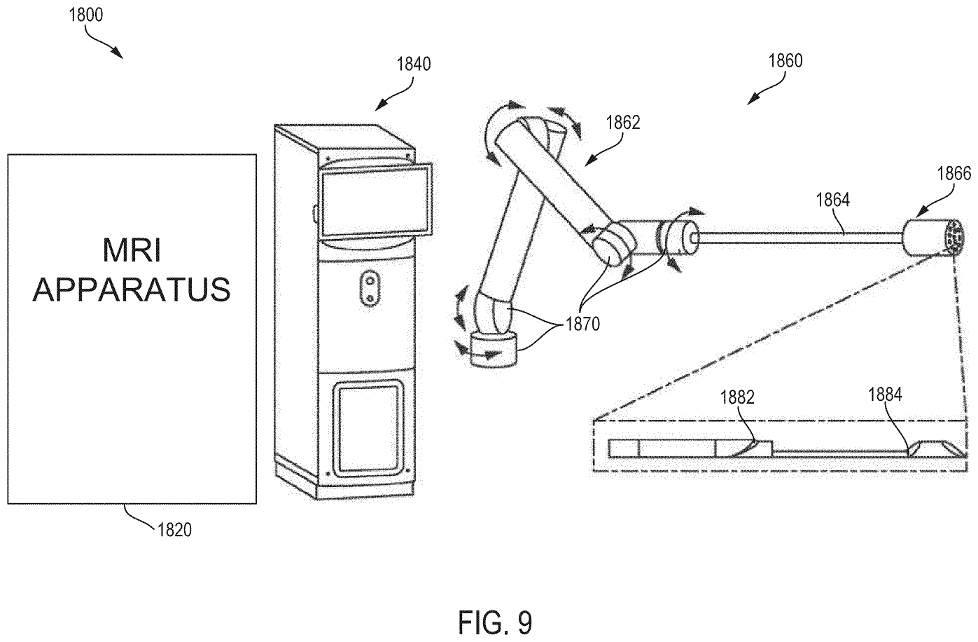

FIG. 9 shows a graphical illustration of a robotic system 1800 that may be used for neural intervention with an access aperture of a Halbach dome, in accordance with various embodiments. The robotic system 1800 includes a magnetic imaging apparatus 1820 with a Halbach dome 400, a computer system 1840, and a robotic system 1860. The example magnetic imaging apparatus 1820 can include an access aperture defined by the Halbach dome 400, to provide access to one or more anatomical parts of a patient being imaged during a medical procedure.

The magnetic imaging apparatus 1820 includes an access aperture throughwhich a robotic arm can extend to reach a patient or target site, in other instances, the magnetic imaging apparatus 1820 can include two or more access apertures. Each access aperture can provide access to the patient and/or surgical site. For example, in instances of multiple access apertures, the multiple access apertures can allow access from different directions and/or proximal locations.

In accordance with various embodiments, the robotic system 1860 is configured to be placed outside the magnetic imaging apparatus 1820. As shown in FIG. 9, the robotic system 1860 can include a robotic arm 1862 that is configured for movements at various angles. In accordance with various embodiments, the robotic arm 1862 includes one or more mechanical arm portions, including a hollow shaft 1864 and an end-effector 1866, that is connected in a configuration to allow the robotic arm 1862 to move, rotate, or swivel at various angles via one or more motion controllers 1870. The double-headed curved arrows in FIG. 9 signify rotational motions produced by the motion controllers 1870.

In accordance with various embodiments, the robotic arm 1862 of the robotic system 1860 is configured for accessing various anatomical parts of interest through or around the magnetic imaging apparatus 1820. In accordance with various embodiments, the access aperture is specifically designed to provide access to the robotic arm 1862 of the robotic system 1860 for operation at various anatomical parts of interest of the patient during a medical procedure, in accordance with various embodiments, the access aperture is designed to account for the size of the robotic arm 1862. For example, the access aperture defines a circumference that is configured to accommodate a robotic arm therethrough, such as the various robotic arms described herein, in accordance with various embodiments, the robotic arm 1862 of the robotic system 1860 is configured for accessing various anatomical parts of the patient from around a side of the magnetic imaging apparatus 1820.

Magnetic imaging apparatuses are further described in U.S. patent application Ser. No. 16/003,585, titled UNILATERAL MAGNETIC RESONANCE IMAGING SYSTEM WITH APERTURE FOR INTERVENTIONS AND METHODOLOGIES FOR OPERATING SAME, filed Jun. 6, 2018, which is incorporated by reference herein in its entirety. The reader will appreciate that the robotic system 1860 can you used in combination with various Halbach domes and cylinders described herein, in certain aspects of the present disclosure.

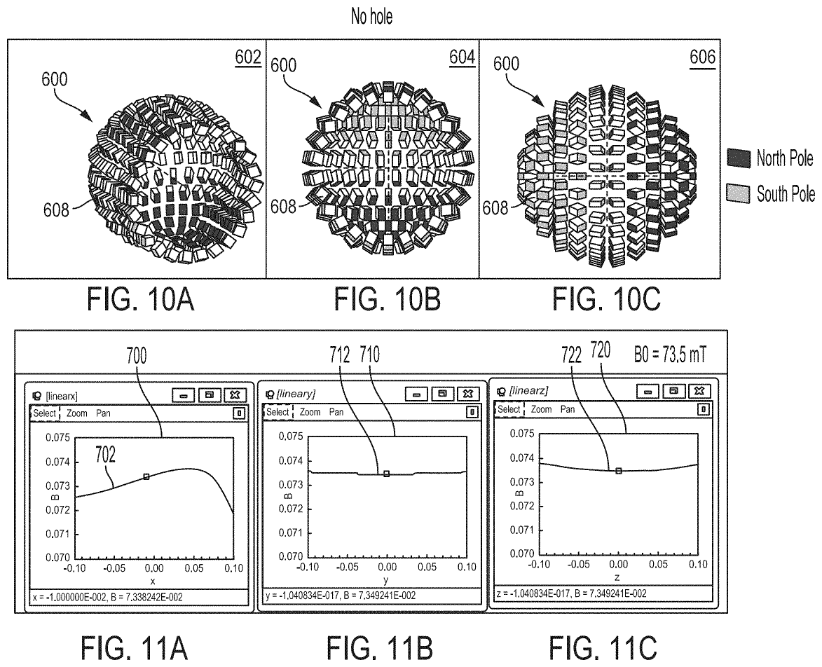

FIGS. 10A-10C illustrate various views of a line simulation of an Halbach dome 600 without an access aperture. The views include an isometric view 602, a bottom view 604, and a top view 606. The Halbach dome 600 comprises a plurality of magnetic elements 608 in a Halbach array. The magnetic elements 608 are shown with a north pole and south pole. The magnetic elements generate a resulting magnetic flux density, B, that points in the direction from the south pole to the north pole, along the Z-axis as shown by the vertical axis in FIG. 10C. FIG. 10A shows the X-axis from the crown of the dome through the center of the base and FIG. 10B shows the Y-axis from along the vertical axis.

FIGS. 11A-11C illustrate magnetic flux density B along the x, y, z directions of the Halbach dome 600 shown in FIGS. 10A-10C. The vertical axis scale ranges from B=70 mT to B=75 mT. A first graph 700 illustrates a magnetic flux density B curve 702 along the x axis, where Bₒ≈73.4 mT at x=0. A second graph 710 illustrates a magnetic flux density B curve 712 along the y axis, where Bₒ≈73.5 mT at y=0. A third graph 720 illustrates a magnetic flux density B curve 722 along the z axis, where Bₒ≈73.4 mT at z=0. Accordingly, nominal magnetic flux density Bₒ≈73.5 mT in this example.

The magnetic flux density B curve 712 along the y axis and the curve 722 along the z axis are relatively flat and maintain a relatively consist magnetic flux density in the predetermined space. In various aspects, the position and orientation of the magnetic elements may be configured to generate a homogeneous magnetic flux density B.

FIGS. 12A-12C illustrate various views of a line simulation of an Halbach dome 800 defining an access aperture 810, including an isometric view 802, a bottom view 804, and a top view 806, in accordance with at least one aspect of the present disclosure. The Halbach dome 800 comprises a plurality of magnetic elements 808 in a Halbach array. The magnetic elements 808 are shown with a north pole and south pole. The magnetic elements generate a resulting magnetic flux density, B, that points in the direction from the south pole to the north pole, along the Z-axis as shown by the vertical axis in FIG. 12C. FIG. 12A shows the X-axis from the crown of the dome through the center of the base and FIG. 12B shows the Y-axis from along the vertical axis. The access aperture 810 has a diameter D≈10 cm.

FIGS. 13A-13C illustrate magnetic flux density B relative to the x, y, z directions of the Halbach dome 800 shown in FIGS. 12A-12C. The vertical axis scale ranges from B=70 mT to B=75 mT. A first graph 900 illustrates a magnetic flux density B curve 902 along the x axis, where Bₒ≈72.5 mT at x=0. A second graph 910 illustrates a magnetic flux density B curve 912 along the y axis, where Bₒ≈72.8 mT at y=0. A third graph 920 illustrates a magnetic flux density B curve 922 along the z axis, where Bₒ≈72.8 mT at z=0. Accordingly, nominal magnetic flux density Bₒ≈72.7 mT in this example.

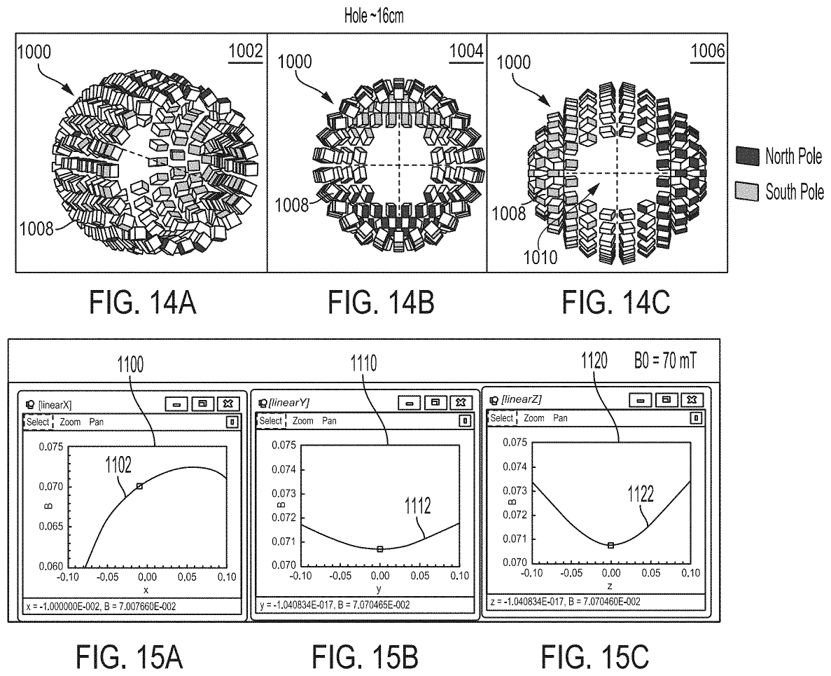

FIGS. 14A-14C illustrate various views of a line simulation of an Halbach dome 1000 defining an access aperture 1010, including an isometric view 1002, a bottom view 1004, and a top view 1006, in accordance with at least one aspect of the present disclosure. The access aperture 1010 has a diameter D≈16 cm. The magnetic elements 1008 generate a resulting magnetic flux density, B, that points in the direction from the south pole to the north pole, along the Z-axis as shown by the vertical axis in FIG. 14C. FIG. 14A shows the X-axis from the crown of the dome through the center of the base and FIG. 14B shows the Y-axis from along the vertical axis.

FIGS. 15A-15C illustrates magnetic flux density B relative to the x, y, z directions of the Halbach dome 1000 shown in FIGS. 14A-14C, and magnetic flux density B along the vertical axis. The vertical axis scale ranges from B=70 mT to B=75 mT. A first graph 1100 illustrates a magnetic flux density B curve 1102 along the x axis, where Bₒ≈70.1 mT at x=0. A second graph 1110 illustrates a magnetic flux density B curve 1112 along the y axis, where Bₒ≈70.7 mT at y=0. A third graph 1120 illustrates a magnetic flux density B curve 1122 along the z axis, where Bₒ≈70.7 mT at z=0. Accordingly, nominal magnetic flux density Bₒ≈70 mT in this example.

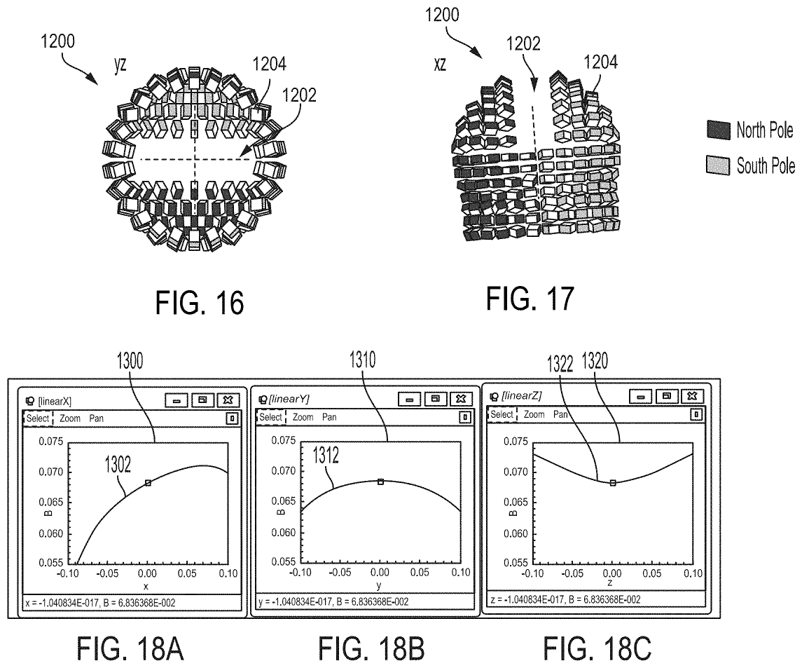

FIG. 16 is a view of a line simulation of an Halbach dome 1200 defining an access gap 1202 on the yz plane and FIG. 17 is a view of the Halbach dome 1200 with the access gap 1202 on the xz plane, in accordance with at least one aspect of the present disclosure. The access gap 1202 is defined according to a structural housing that supports a plurality of magnetic elements 1204 in the Halbach dome 1200. The magnetic elements 1204 are shown with a north pole and south pole. The access gap 1202 has a width W_{gap}≈10 cm, roughly equivalent to a closed Halbach dome configuration that has 10 cubic magnetic elements removed along the width of a gap, W_{gap}.

FIGS. 18A-18C illustrate magnetic flux density B relative to the x, y, z directions a of the Halbach dome 1200 shown in FIGS. 16 and 17. The vertical axis scale ranges from B=70 mT to B=75 mT. A first graph 1300 illustrates a magnetic flux density B curve 1302 along the x axis, where Bₒ≈68.4 mT at x=0. A second graph 1310 illustrates a magnetic flux density B curve 1312 along the y axis, where Bₒ≈68.4 mT at y=0. A third graph 1320 illustrates a magnetic flux density B curve 1322 along the z axis, where Bₒ≈68.4 mT at z=0. Accordingly, nominal magnetic flux density Bₒ≈68.5 mT in this example.

FIGS. 10A-18C demonstrate variations in different Halbach dome configurations and their respective impact to the magnetic flux density. The different configurations demonstrated a surprisingly small impact to the overall magnetic flux density within a particular volume while providing access to a patient though the access apertures.

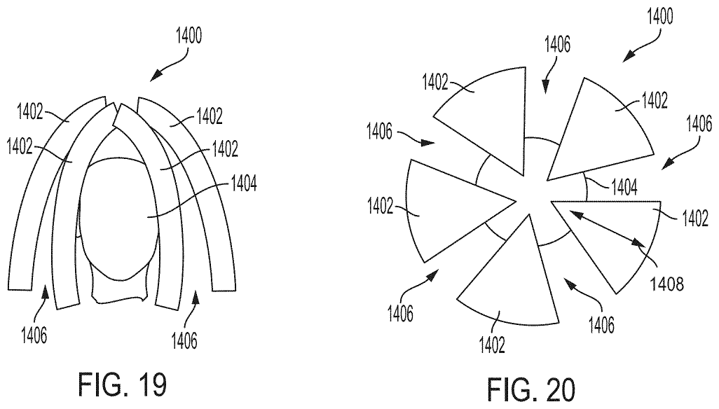

FIG. 19 is a schematic illustration of an Halbach dome 1400 comprising a plurality of wedges 1402, in accordance with at least one aspect of the present disclosure.

FIG. 20 is a top down view of the Halbach dome 1400 comprising the wedges 1402 shown in FIG. 19, in accordance with at least one aspect of the present disclosure. The wedges 1402 can be positioned to surround the patient's head 1404 and access apertures 1406 defined between the wedges 1404 are configured to allow access to the patient's head 1404 to enable neural intervention. In various aspect, the wedges are structural components that comprise a plurality of magnetic elements in a Halbach array. In one aspect, a wedge 1402 may be removed to provide access to the patient's head 1404 for neural intervention. In one aspect, each wedge may be configured to move along a respective radial axis 1408 towards the center of the patient's head 1404 or away from the center of the patient's head 1404. As the wedges 1402 are moved away from the center of the patient's head, the access apertures 1406 increase in size. As the wedges 1402 are moved towards the center of the patient's head, the access apertures 1406 decrease in size until they gaps between the wedges are sealed. Each wedge 1402 may be moved individually or proportionally with the movement of the other wedges 1402.

FIG. 21 is an isometric view of an Halbach dome 1500 defining a plurality of access apertures 1502, in accordance with at least one aspect of the present disclosure.

FIG. 22 is an isometric view of an Halbach dome 1600 defining a plurality of access apertures 1602 and an adjustable gap 1604, in accordance with at least one aspect of the present disclosure. Halbach dome 1600 comprises a bonding agent 1606 such as an epoxy resin that holds the plurality of magnetic elements in a fixed position. The plurality of magnetic elements 1608 are bounded to a structural housing 1610, such as a plastic substrate, for example. In various aspects, the bonding agent 1606 and structural housing 1610 may be non-conductive or diamagnetic materials. In the present aspect, the Halbach dome 1600 comprises two structural housings 1610. In various aspect, a Halbach dome may comprise more than two structural housings, such as in FIGS. 19 and 20, for example. The access apertures 1602 in the structural housing 1610 provide a passage directly to the patient and are not obstructed by the structural housing 1610, bonding agent 1606, or magnetic elements 1608. The location of the access apertures 1602 may be selected or configured in an open space in the magnetic assembly configuration.

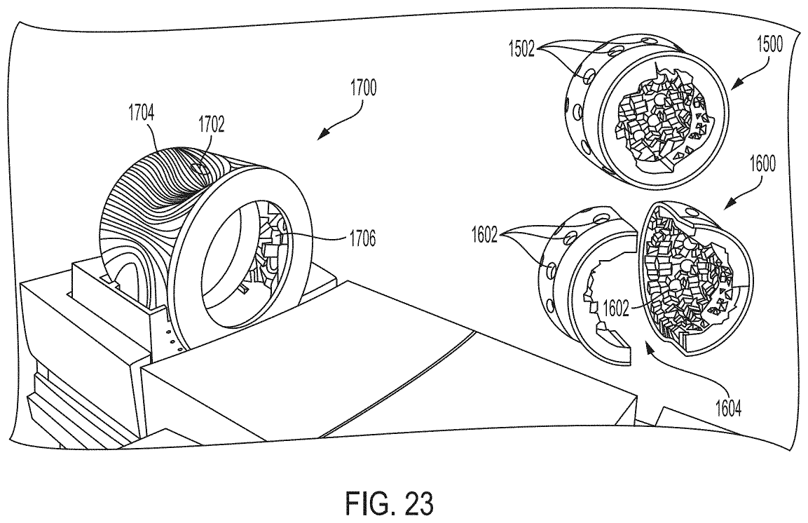

FIG. 23 illustrates a scanning system 1700 comprising a Halbach dome 1702 defining an access aperture (not shown in this view), in accordance with at least one aspect of the present disclosure. In one aspect, the scanning system 1700 may be outfitted with an optional Halbach dome 1500 defining a plurality of access apertures 1502 as described in connection with FIG. 21, for example. In one aspect, the scanning system 1700 may be outfitted with an Halbach dome 1600 defining a plurality of access apertures 1602 and defining an adjustable gap 1604 as described in connection with FIG. 22, for example. The scanning system 1700 may comprise gradient coils 1704 on the exterior of the Halbach dome. Additionally, the interior of the Halbach dome comprises shim magnets 1706 in a shim tray that allows a technician to granularly configure the magnetic flux density of the Halbach dome.

Various magnetic dome structures described herein can be utilized with a MRI system as show in FIG. 4. The MRI system can include an auxiliary cart that houses the electrical and electronic components, such as a computer, programmable logic controller, power distribution unit, and amplifiers, for example. The MRI system can also include a magnet cart that houses the magnetic dome structure, gradient coils, and transmission coil and attaches to the receive coil. Various additional aspects of the subject matter described herein are set out in the following numbered examples:

Example 1: A magnetic resonance imaging apparatus comprising: a structural housing configured as a dome shape, wherein the structural housing is configured to receive a patient's head at the base of the dome, and wherein the structural housing defines an access aperture configured to allow access to the patient's head to enable neural intervention; and a plurality of magnetic elements configured in a Halbach array, wherein the plurality of magnetic elements are permanently affixed to an interior surface of the structural housing.

Example 2: The magnetic resonance imaging apparatus of Example 1, wherein the access aperture is configured in the form of a hole defining a diameter.

Example 3: The magnetic resonance imaging apparatus of Example 2, wherein the diameter of the hole is adjustable.

Example 4: The magnetic resonance imaging apparatus of Examples 1-3, wherein the access aperture is configured in the form of a gap defining a width.

Example 5: The magnetic resonance imaging apparatus of Example 4, wherein the width of the gap is adjustable.

Example 6: The magnetic resonance imaging apparatus of Examples 1-5, comprising a plurality of access apertures.

Example 7: The magnetic resonance imaging apparatus of Example 6, wherein each one of the plurality of access apertures is in the form of a hole.

Example 8: The magnetic resonance imaging apparatus of Example 6, wherein each one of the plurality of access apertures is in the form of a gap.

Example 9: The magnetic resonance imaging apparatus of Example 8, wherein the width of each one of the plurality of access aperture gaps are adjustable.

Example 10: The magnetic resonance imaging apparatus of Examples 1-9, comprising a plurality of access apertures, wherein at least one access aperture is in the form of a hole and at least one access aperture is in the form of a gap.

Example 11: The magnetic resonance imaging apparatus of Examples 1-10, wherein the structural housing is configured to rotate such that the access aperture aligns with a target location for neural intervention.

Example 12: A magnetic resonance imaging apparatus comprising: a structural housing configured as a dome shape, wherein the structural housing is configured to receive a patient's head at the base of the dome; the structural housing comprises a plurality of wedges defining an access aperture configured to allow access to the patient's head to enable neural intervention; and a plurality of magnetic elements configured in a Halbach array, wherein the plurality of magnetic elements are permanently affixed to an interior surface of the structural housing.

Example 13: The magnetic resonance imaging apparatus of Example 12, wherein the access aperture is configured in the form of a gap defining a width between at least two of the plurality of wedges.

Example 14: The magnetic resonance imaging apparatus of Example 13, wherein the width of the gap is adjustable.

Example 15: The magnetic resonance imaging apparatus of Example 13, wherein the width of the gap is adjustable by moving at least one of the plurality of wedges towards the center of the dome shape or away from the center of the dome shape along a longitudinal axis.

Example 16: The magnetic resonance imaging apparatus of Examples 12-15, comprising a plurality of access apertures.

Example 17: The magnetic resonance imaging of Example 12-16, wherein at least one of the plurality of wedges is removable to allow access to the patient's head to enable neural intervention.

Example 18: The magnetic resonance imaging apparatus of Example 12-17, wherein the structural housing is configured to rotate such that the access aperture aligns with a target location for neural intervention.

Example 19: The magnetic resonance imaging apparatus of Example 12-18, comprising a plurality of access apertures, wherein at least one access aperture is in the form of a gap and at least one access aperture is in the form of a hole defining a diameter by the structural housing.

Example 20: A neural intervention system, comprising: a magnetic resonance imaging system comprising: a magnetic resonance imaging apparatus further comprising a plurality of magnetic elements in Halbach dome, wherein the Halbach dome defines an access aperture configured to allow access to the patient's head to enable neural intervention; and a guided robotic system comprising: a robotic arm wherein the guided robotic system is configured to guide the robotic arm through the access aperture of the Halbach dome for neural intervention.

Example 21: A magnetic resonance imaging apparatus comprising: a cylindrical structural housing configured to receive a patient's head and defining an access aperture and configured to allow access to the patient's head to enable neural intervention, wherein the access aperture is defined in the wall structure of the Halbach cylinder; and a plurality of magnetic elements configured in a Halbach array, wherein the plurality of magnetic elements are permanently affixed to an interior surface of the structural housing.

Example 22: The magnetic resonance imaging apparatus of Example 21, wherein the access aperture is configured in the form of a hole defining a diameter.

Example 23: The magnetic resonance imaging of Example 22, wherein the diameter of the hole is adjustable.

Example 24: The magnetic resonance imaging of Example 21-23, wherein the access aperture is configured in the form of a gap defining a width.

Example 25: The magnetic resonance imaging of Example 24, wherein the width of the gap is adjustable.

Example 26: The magnetic resonance imaging of Example 21-25, comprising a plurality of access apertures.

Example 27: The magnetic resonance imaging of Example 26, wherein each one of the plurality of access apertures is in the form of a hole.

Example 28: The magnetic resonance imaging of Example 26, wherein each one of the plurality of access apertures is in the form of a gap.

Example 29: The magnetic resonance imaging of Example 21-28, comprising a plurality of access apertures, wherein at least one access aperture is in the form of a hole and at least one access aperture is in the form of a gap.

While several forms have been illustrated and described, it is not the intention of Applicant to restrict or limit the scope of the appended claims to such detail. Numerous modifications, variations, changes, substitutions, combinations, and equivalents to those forms may be implemented and will occur to those skilled in the art without departing from the scope of the present disclosure. Moreover, the structure of each element associated with the described forms can be alternatively described as a means for providing the function performed by the element. Also, where materials are disclosed for certain components, other materials may be used. It is therefore to be understood that the foregoing description and the appended claims are intended to cover all such modifications, combinations, and variations as falling within the scope of the disclosed forms. The appended claims are intended to cover all such modifications, variations, changes, substitutions, modifications, and equivalents.

The foregoing detailed description has set forth various forms of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, and/or examples can be implemented, individually and/or collectively, by a wide range of hardware, software, firmware, or virtually any combination thereof. Those skilled in the art will recognize that some aspects of the forms disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as one or more program products in a variety of forms, and that an illustrative form of the subject matter described herein applies regardless of the particular type of signal bearing medium used to actually carry out the distribution.

Instructions used to program logic to perform various disclosed aspects can be stored within a memory in the system, such as dynamic random access memory (DRAM), cache, flash memory, or other storage. Furthermore, the instructions can be distributed via a network or by way of other computer readable media. Thus a machine-readable medium may include any mechanism for storing or transmitting information in a form readable by a machine (e.g., a computer), but is not limited to, floppy diskettes, optical disks, compact disc, read-only memory (CD-ROMs), and magneto-optical disks, read-only memory (ROMs), random access memory (RAM), erasable programmable read-only memory (EPROM), electrically erasable programmable read-only memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the non-transitory computer-readable medium includes any type of tangible machine-readable medium suitable for storing or transmitting electronic instructions or information in a form readable by a machine (e.g., a computer).

As used in any aspect herein, the term “control circuit” may refer to, for example, hardwired circuitry, programmable circuitry (e.g., a computer processor including one or more individual instruction processing cores, processing unit, processor, microcontroller, microcontroller unit, controller, digital signal processor (DSP), programmable logic device (PLD), programmable logic array (PLA), or field programmable gate array (FPGA)), state machine circuitry, firmware that stores instructions executed by programmable circuitry, and any combination thereof. The control circuit may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc. Accordingly, as used herein “control circuit” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof

As used in any aspect herein, the term “logic” may refer to an app, software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

As used in any aspect herein, the terms “component,” “system,” “module” and the like can refer to a control circuit computer-related entity, either hardware, a combination of hardware and software, software, or software in execution.

As used in any aspect herein, an “algorithm” refers to a self-consistent sequence of steps leading to a desired result, where a “step” refers to a manipulation of physical quantities and/or logic states which may, though need not necessarily, take the form of electrical or magnetic signals capable of being stored, transferred, combined, compared, and otherwise manipulated. It is common usage to refer to these signals as bits, values, elements, symbols, characters, terms, numbers, or the like. These and similar terms may be associated with the appropriate physical quantities and are merely convenient labels applied to these quantities and/or states.

A network may include a packet switched network. The communication devices may be capable of communicating with each other using a selected packet switched network communications protocol. One example communications protocol may include an Ethernet communications protocol which may be capable permitting communication using a Transmission Control Protocol/Internet Protocol (TCP/IP). The Ethernet protocol may comply or be compatible with the Ethernet standard published by the Institute of Electrical and Electronics Engineers (IEEE) titled “IEEE 802.3 Standard”, published in December, 2008 and/or later versions of this standard. Alternatively or additionally, the communication devices may be capable of communicating with each other using an X.25 communications protocol. The X.25 communications protocol may comply or be compatible with a standard promulgated by the International Telecommunication Union-Telecommunication Standardization Sector (ITU-T). Alternatively or additionally, the communication devices may be capable of communicating with each other using a frame relay communications protocol. The frame relay communications protocol may comply or be compatible with a standard promulgated by Consultative Committee for International Telegraph and Telephone (CCITT) and/or the American National Standards Institute (ANSI). Alternatively or additionally, the transceivers may be capable of communicating with each other using an Asynchronous Transfer Mode (ATM) communications protocol. The ATM communications protocol may comply or be compatible with an ATM standard published by the ATM Forum titled “ATM-MPLS Network Interworking 2.0” published August 2001, and/or later versions of this standard. Of course, different and/or after-developed connection-oriented network communication protocols are equally contemplated herein.

Unless specifically stated otherwise as apparent from the foregoing disclosure, it is appreciated that, throughout the foregoing disclosure, discussions using terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

One or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

The terms “proximal” and “distal” are used herein with reference to a clinician manipulating the handle portion of the surgical instrument. The term “proximal” refers to the portion closest to the clinician and the term “distal” refers to the portion located away from the clinician. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flow diagrams are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures or characteristics may be combined in any suitable manner in one or more aspects.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

What is claimed is:

1. A magnetic resonance imaging apparatus comprising:
a structural housing configured as a dome shape, wherein the structural housing is configured to receive a patient's head at the base of the dome, and wherein the structural housing defines an access aperture configured to allow access to the patient's head to enable neural intervention; and
a plurality of magnetic elements configured in a Halbach array, wherein the plurality of magnetic elements are permanently affixed to an interior surface of the structural housing.

2. The magnetic resonance imaging apparatus of claim 1, wherein the access aperture is configured in the form of a hole defining a diameter.

3. The magnetic resonance imaging apparatus of claim 2, wherein the diameter of the hole is adjustable.

4. The magnetic resonance imaging apparatus of claim 1, wherein the access aperture is configured in the form of a gap defining a width.

5. The magnetic resonance imaging apparatus of claim 4, wherein the width of the gap is adjustable.

6. The magnetic resonance imaging apparatus of claim 1, comprising a plurality of access apertures.

7. The magnetic resonance imaging apparatus of claim 6, wherein each one of the plurality of access apertures is in the form of a hole.

8. The magnetic resonance imaging apparatus of claim 6, wherein each one of the plurality of access apertures is in the form of a gap.

9. The magnetic resonance imaging apparatus of claim 8, wherein the width of each one of the plurality of access aperture gaps are adjustable.

10. The magnetic resonance imaging apparatus of claim 1, comprising a plurality of access apertures, wherein at least one access aperture is in the form of a hole and at least one access aperture is in the form of a gap.

11. The magnetic resonance imaging apparatus of claim 1, wherein the structural housing is configured to rotate such that the access aperture aligns with a target location for neural intervention.

12. A magnetic resonance imaging apparatus comprising:
a structural housing configured as a dome shape, wherein the structural housing is configured to receive a patient's head at the base of the dome;
the structural housing comprises a plurality of wedges defining an access aperture configured to allow access to the patient's head to enable neural intervention; and
a plurality of magnetic elements configured in a Halbach array, wherein the plurality of magnetic elements are permanently affixed to an interior surface of the structural housing.

13. The magnetic resonance imaging apparatus of claim 12, wherein the access aperture is configured in the form of a gap defining a width between at least two of the plurality of wedges.

14. The magnetic resonance imaging apparatus of claim 13, wherein the width of the gap is adjustable.

15. The magnetic resonance imaging apparatus of claim 13, wherein the width of the gap is adjustable by moving at least one of the plurality of wedges towards the center of the dome shape or away from the center of the dome shape along a longitudinal axis.

16. The magnetic resonance imaging apparatus of claim 12, comprising a plurality of access apertures.

17. The magnetic resonance imaging of claim 12, wherein at least one of the plurality of wedges is removable to allow access to the patient's head to enable neural intervention.

18. The magnetic resonance imaging apparatus of claim 12, wherein the structural housing is configured to rotate such that the access aperture aligns with a target location for neural intervention.

19. The magnetic resonance imaging apparatus of claim 12, comprising a plurality of access apertures, wherein at least one access aperture is in the form of a gap and at least one access aperture is in the form of a hole defining a diameter by the structural housing.

20. A neural intervention system, comprising:
a magnetic resonance imaging system comprising:
a magnetic resonance imaging apparatus further comprising a plurality of magnetic elements in Halbach dome, wherein the Halbach dome defines an access aperture configured to allow access to the patient's head to enable neural intervention; and
a guided robotic system comprising: a robotic arm wherein the guided robotic system is configured to guide the robotic arm through the access aperture of the Halbach dome for neural intervention.
